(19) United States
(12) Patent Application Publication
(10) Pub. No.: US 2026/0344981 A1
(43) Pub. Date: Nov. 5, 2026

(54) Endoscope and Associated Electrical Connection

(71) Applicant: KARL STORZ SE & Co. KG, Tuttlingen (DE)

(72) Inventors: Klaus Storz, VS-Schwenningen (DE); Klaus Roth, Frittlingen (DE)

(73) Assignee: KARL STORZ SE & Co. KG, Tuttlingen (DE)

(21) Appl. No.: 19/483,947

(22) PCT Filed: Mar. 26, 2024
(86) PCT No.: PCT/EP2024/058166

§ 371 Date: Nov. 12, 2025

(30) Foreign Application Priority Data
Mar. 28, 2023 (DE) 10 2023 107 813.5

(51) Int. Cl. A61B 1/00

(52) U.S. Cl. CPC A61B 1/00124; A61B 1/00114

(57) ABSTRACT

The invention relates to an endoscope device (10) comprising: a shaft (12) with a cable feedthrough (14) which extends from a proximal side (16) of the shaft (12) to a distal side (18) of the shaft (12); a flat cable (20) which extends through the cable feedthrough (14) from the proximal side (16) to the distal side (18), wherein the flat cable (20) comprises a contact section (24) with at least three cable contacts (26) on the proximal side (16), and the cable contacts (26) are arranged one behind the other with respect to the longitudinal axis of the flat cable (20); and a socket (28) which is arranged on the proximal side (16) of the shaft (12) and which comprises a cable receiving area (30) that is designed to receive the contact section (24) of the flat cable (20). The cable receiving area (30) has at least three socket contacts (32) which are arranged one behind the other with respect to the longitudinal axis of the cable receiving area (30) and so as to correspond to the cable contacts (26).

The invention additionally relates to an electric connector, to a flat cable (20), to a socket (28), and to a method for assembling an endoscope device.

Endoscopes with a shaft are known from the prior art which have an electrical connection with a cable between a distal side of the shaft and a proximal side of the shaft to transmit data and/or energy. For example, a light source can be arranged on the distal side and is supplied with electrical energy from the proximal side via the cable. In particular with video endoscopes, data from an image sensor attached to the distal side of the shaft must also be transmitted to the proximal side of the shaft for image generation in order to further process the generated image data. In addition, the image sensor in such video endoscopes needs to be supplied with energy to generate the image. Typically, a flat cable that runs through a cable guide in the shaft is used for data and power transmission.

Such flat cables are limited in width by the dimensions of the cable routing of the shaft and may therefore comprise a limited number of conductor tracks. Although the number of conductor tracks can be increased by reducing the distances between adjacent conductor tracks, connecting the closely spaced conductor tracks to a socket or a circuit board is associated with a high level of effort. Typically, a ZIF connector (zero-insertion-force connector) is used for a simple connection, providing a low profile and light weight, as well as a secure and detachable connection if required.

Such ZIF connectors require a specific surface dimension for the individual conductor tracks in order to be attached to the flat cable. Flat cables are also known that have enlarged contact sections at their ends, so that a ZIF connector can be attached to them.

When the endoscope device is assembled, the flat cable must be fed through the shaft of the endoscope. Corresponding shafts have cable feedthroughs that extend through the shaft in question. The size of such cable feedthroughs is limited by the diameter of the relevant shaft. In practice, an end with contact sections for a ZIF connector defines a maximum width of the flat cable and thus a number of conductor tracks. With a large number of conductor tracks, a cable feedthrough with a corresponding width and therefore a correspondingly large shaft diameter is required.

Based on the prior art, the invention aims to achieve a high degree of flexibility with regard to possible designs of an endoscope device as well as simple manufacturability.

This object is achieved according to the invention by an endoscope device, an endoscope, an electrical connector, a flat cable, a socket and a method for assembling an endoscope device as described herein and defined in the claims.

An endoscope device may be provided that comprises a shaft with a cable feedthrough, a flat cable and a socket. The cable feedthrough of the shaft extends from a proximal side of the shaft to a distal side of the shaft. The flat cable extends through the cable feedthrough from the proximal side to the distal side. The flat cable comprises a contact section with at least three cable contacts on the proximal side. The cable contacts are arranged one behind the other with respect to a longitudinal axis of the flat cable. The socket is located on the proximal side of the shaft and comprises a cable receiving area. The cable receiving area is designed to accommodate the contact section of the flat cable. The cable receiving area has at least three socket contacts, which are arranged one behind the other with respect to a longitudinal axis of the cable receiving area and so as to correspond to the cable contacts.

Furthermore, an endoscope may be provided, which includes the endoscope device.

Furthermore, an electrical connector can be provided for an endoscope device, in particular for an endoscope device according to the invention. The connector comprises a flat cable which comprises a contact section with at least three cable contacts, wherein the cable contacts are arranged one behind the other with respect to a longitudinal axis of the flat cable. Furthermore, the connector comprises a socket which comprises a cable receiving area designed to receive the contact section of the flat cable, wherein the cable receiving area has at least three socket contacts which are arranged one behind the other with respect to a longitudinal axis of the cable receiving area and so as to correspond to the cable contacts.

Furthermore, a flat cable may be provided, in particular a flat cable for a connector according to the invention. The flat cable comprises a contact section with at least three cable contacts, wherein the cable contacts are arranged one behind the other with respect to a longitudinal axis of the flat cable.

Furthermore, a socket for a flat cable can be provided, in particular a socket for a flat cable according to the invention and/or for a connector according to the invention. The socket comprises a cable receiving area which is designed to receive a contact section of the flat cable, in particular of a flat cable according to the invention, wherein the cable receiving area has at least three socket contacts which are arranged one behind the other with respect to a longitudinal axis of the cable receiving area.

Furthermore, a method for assembling an endoscope device may be provided, in particular a method for assembling an endoscope device according to the invention. The method comprises providing a shaft with a cable feedthrough extending from a proximal side of the shaft to a distal side of the shaft. Furthermore, the method comprises providing a flat cable, wherein the flat cable comprises on the proximal side a contact section with at least three cable contacts, the cable contacts being arranged one behind the other with respect to a longitudinal axis of the flat cable. The method further comprises providing a socket on the proximal side of the shaft, wherein the socket comprises a cable receiving area which is designed to receive the contact section of the flat cable, wherein the cable receiving area has at least three socket contacts arranged one behind the other with respect to a longitudinal axis of the cable receiving area and so as to correspond to the cable contacts. Furthermore, the method comprises passing the flat cable through the cable feedthrough. Furthermore, the method comprises inserting the contact section of the flat cable into the cable receiving area of the socket.

The features according to the invention allow for a high degree of flexibility regarding possible designs of an endoscope device as well as easy manufacturability. An endoscope device can be provided which can transmit a high amount of data and/or energy between a proximal side and a distal side. A high degree of structural compactness and/or simplicity can be achieved. In particular, the flat cable can have a high number of conductor tracks and still be narrow. The flat cable can be easily provided with a socket by arranging cable contacts and cable receiving areas one behind the other with respect to their longitudinal axes. Because the cable contacts are arranged one behind the other, a surface area of the cable contacts for attaching the socket can be increased without increasing the width of the flat cable. Even a multi-core flat cable can thus be easily and quickly routed through a narrow cable feedthrough and subsequently connected. The inventors have recognized in particular that the arrangement of contacts one behind the other represents a reliable, simple and efficient alternative to a manufacturing process in which a flat cable is first passed through a cable feedthrough and then provided with a connector, for example by soldering, which is wider than the cable feedthrough.

The endoscope device can be designed for a rigid endoscope. The endoscope device can be designed for a flexible endoscope. The endoscope device must be designed for a video endoscope.

The shaft is designed in particular to be inserted into the interior of organisms or into technical cavities. The distal side of the shaft and the proximal side of the shaft can be opposite each other with respect to a longitudinal axis of the shaft. A longitudinal axis of the shaft can be oriented parallel to the longitudinal axis of the flat cable and/or parallel to the longitudinal axis of the cable receiving area. The shaft can be designed to be inserted with its distal side first. The shaft can be rigid. For example, it can be a rigid endoscope shaft. The shaft can be flexible. For example, it can be a flexible endoscope shaft. The shaft can have a flexible distal end and, in particular, a rigid portion adjacent to the flexible distal end, for example, a rigid proximal and/or middle portion. The cable feedthrough through the shaft runs in particular inside the shaft. The cable feedthrough extends in particular parallel to the longitudinal axis of the shaft.

The cable feedthrough can have an inner width and an inner height, wherein the inner width is at least as great, and in particular at least 2 times greater and preferably at least 3 times greater than the inner height. The inner width of the cable feedthrough can be at most 2.3 mm, preferably at most 1.8 mm, and particularly preferably at most 1.2 mm. The inner height of the cable feedthrough can be at most 1000 μm, preferably at most 700 μm and particularly preferably at most 400 μm. By using a suitably compact cable feedthrough, space can be created even in a small shaft for further components such as working channels, power transmission elements such as wires and/or structural elements such as stiffening elements.

The shaft may have at least one stiffening element that extends parallel to the longitudinal axis of the shaft. At least one stiffening element can in particular be designed to stiffen the shaft and/or to hold the cable feedthrough in position. The shaft can be designed to accommodate Bowden cables inside, in particular inside the at least one stiffening element and/or inside a cable guide. The shaft may comprise a structural element that extends in the longitudinal direction of the shaft. The structural element can be located particularly inside the shaft. The structural element can be designed as a working channel. The structural element can be designed as an additional stiffening element. The structural element can be designed as a light guide. The structural element can be designed as a working channel. The structural element can be designed as a lens system.

The flat cable can be a ribbon cable. The flat cable can be a foil cable (flat flex cable). The flat cable can be a flexible printed circuit board. The flat cable can be, in particular, an FPC cable (flexible printed circuit cable). Such designs of the flat cable can result in low manufacturing costs. Furthermore, components available as mass-produced goods can be used, thus ensuring a reliable availability of said components. The flat cable can be designed to transmit data from a digital image sensor, for example via a MIPI interface of version CSI-2 or CSI-3. The flat cable can be a cable with multiple conductor tracks. The multiple conductor tracks can run parallel to each other. The multiple conductor tracks can extend from each of the cable contacts away from the contact section along the longitudinal axis of the flat cable. The flat cable can be multi-layered and have a plurality of cable layers. The conductor tracks can run in different cable layers. In particular, a plurality of conductor tracks can run in different cable layers.

The contact section of the flat cable is in particular designed to electrically connect the flat cable to a socket, a circuit board and/or a further component. The contact section of the flat cable can have at least four, in particular at least eight and preferably at least twelve cable contacts. This allows a large number of different signals to be transmitted, meaning that, for example, a camera or even a stereo camera and/or one or more light elements can be powered and/or controlled via the flat cable. The contact section can have a width that is less than or equal to the inner width of the cable feedthrough. The contact section can have a height that is less than or equal to the inner height of the cable feedthrough. The width of the contact section can be at most 2.2 mm, preferably at most 1.5 mm, particularly preferably at most 1.2 mm. The height of the contact section can be at most 200 μm, preferably at most 150 μm and particularly preferably at most 70 μm. The contact section can be designed to change the direction of conductor tracks from a direction parallel to the longitudinal axis of the flat cable to a different direction, in particular a direction perpendicular to the longitudinal axis of the flat cable. The contact section may, in particular, have curved conductor tracks.

The flat cable can be designed as a multi-row flat cable, in particular a two-row or three-row flat cable. The contact section of the multi-row flat cable is designed with multiple rows, in particular two or three rows. The contact section of the multi-row flat cable comprises a plurality of cable contacts, wherein at least three cable contacts are arranged one behind the other with respect to a longitudinal axis of the multi-row flat cable. The contact section of the two-row flat cable comprises at least four cable contacts, wherein at least three cable contacts arranged one behind the other with respect to the longitudinal direction of the two-row flat cable form one row of the two rows. The contact section of the three-row flat cable comprises at least five cable contacts, wherein at least three cable contacts arranged one behind the other with respect to the longitudinal direction of the three-row flat cable form one row of the three rows. The contact section of the two-row flat cable may, for example, comprise six cable contacts, wherein the cable contacts are arranged one behind the other with respect to a longitudinal axis of the two-row flat cable in two rows of three cable contacts each. The contact section of the three-row flat cable may, for example, comprise nine cable contacts, wherein the cable contacts are arranged one behind the other with respect to a longitudinal axis of the three-row flat cable in three rows of three cable contacts each. The contact section of the multi-row flat cable is designed to electrically connect the multi-row flat cable to a multi-row socket.

The cable contacts are in particular designed to provide an electrical connection to the conductor tracks. The cable contacts are arranged in an offset manner, in particular along the longitudinal axis of the flat cable. The cable contacts can be spaced apart, in particular in the longitudinal direction of the flat cable. The cable contacts can each have an area of at least 0.10 mm², preferably at least 0.25 mm² and particularly preferably at least 0.40 mm². The cable contacts can each have an area of at most 0.40 mm², preferably at most 0.25 mm², and particularly preferably at most 0.10 mm². The surface of the cable contacts is, in particular, a surface area that is available for an electrical connection. A distance between adjacent cable contacts can preferably be at least 0.20 mm, preferably at least 0.40 mm and particularly preferably at least 0.60 mm. A distance between adjacent cable contacts can be at most 0.60 mm, preferably at most 0.40 mm, and most preferably at most 0.20 mm.

The socket is in particular designed to provide an electrical connection to the cable contacts and to make this available for a circuit board and/or further components. The cable receiving area of the socket can have at least four, in particular at least eight and preferably at least twelve socket contacts. The number of socket contacts can correspond to the number of cable contacts. The socket contacts can be arranged offset in a longitudinal direction of the socket. The socket contacts can be spaced apart, in particular in a longitudinal direction of the socket. The socket contacts can be arranged in the same pattern as the cable contacts.

The socket can be designed as a multi-row socket, in particular a two-row or three-row socket. The cable receiving area of the multi-row socket is designed with multiple rows, in particular two or three rows. The cable receiving area of the multi-row socket comprises a plurality of socket contacts, wherein at least three socket contacts are arranged one behind the other with respect to a longitudinal axis of the multi-row socket. The cable receiving area of the two-row socket comprises at least four socket contacts, wherein at least three socket contacts arranged one behind the other with respect to the longitudinal direction of the two-row socket form one row of the two rows. The cable receiving area of the three-row socket comprises at least five socket contacts, wherein at least three socket contacts arranged one behind the other with respect to the longitudinal direction of the three-row socket form one row of the three rows. The cable receiving area of the two-row socket may, for example, comprise six socket contacts, wherein the socket contacts are arranged one behind the other with respect to a longitudinal axis of the two-row socket in two rows of three socket contacts each. The cable receiving area of the three-row socket may, for example, comprise nine socket contacts, wherein the socket contacts are arranged one behind the other with respect to a longitudinal axis of the three-row socket in three rows of three socket contacts each. The cable receiving area of the multi-row socket can be designed to accommodate the contact section of the multi-row flat cable, with the socket contacts being arranged according to the cable contacts.

The cable receiving area may comprise a first positioning element, and the contact section of the flat cable may comprise a second positioning element. The first positioning element and the second positioning element can be designed to work together to position the contact section in the cable receiving area, at least with respect to the longitudinal axis of the cable receiving area. As a result, an assembly can be simplified. Furthermore, errors during assembly can be prevented. The positioning elements can be part of a positioning aid and/or form a positioning aid. The first positioning element can include one or more positioning pins. The second positioning element may comprise a receptacle designed to accommodate the one or more positioning pins. The receptacle can be, in particular, one or more feedthroughs. The first positioning element can include one or more positioning ribs. The second positioning element may comprise one or more notches designed to accommodate one or more positioning ribs. The first positioning element may comprise one or more retaining lugs. The second positioning element may comprise one or more guide grooves, which are designed to accommodate one or more retaining lugs.

The cable receiving area can have a base and two side walls. The two side walls can together define a receiving space for the contact section of the flat cable. The side walls can each extend parallel to a longitudinal axis of the cable receiving area. The socket device can comprise a lid that is designed to close off the cable receiving area. The lid can be designed to exert a force on the contact section in the closed state, pressing the cable contacts against the socket contacts. The socket can be designed to allow the cable contacts to be inserted transversely and/or obliquely to a longitudinal axis of the socket.

The socket contacts can be designed as spring contacts. The spring contacts can be formed in particular as spring pins, contact springs, and/or contact pads. The lid and the cable receiving area can be slid into each other to close off the cable receiving area. The lid can have at least one lid projection and the cable receiving area can have at least one receptacle recess, wherein the at least one lid projection and the at least one receptacle recess can be slid into each other to close off the cable receiving area. Preferably, the lid and/or the cable receiving area can have an insertion bevel. This makes inserting the lid during the assembly of the socket easy and gentle. Alternatively or additionally, the lid and/or cable receiving area can have an end stop. This ensures that the closed state can be reliably achieved. The lid and cable receiving area may have a locking mechanism. The locking mechanism may comprise a locking projection and a locking recess that interlock in the closed state. The locking mechanism prevents movement from the closed state, and thus allows secure fixing.

The cable receiving area and the lid can be connected via a pivot joint. This provides for easy assembly. The pivot joint can be a detachable pivot joint. The pivot joint is preferably a hinge. The hinge can be formed by pins that are rotatably inserted into openings. The detachable hinge can be formed by flattened pins that can be removed from the openings. The detachable connection allows for easy replacement of the lid in case of a malfunction.

The lid and cable receiving area can have a snap connection designed to exert a force on the contact section in the closed state, thereby pressing the cable contacts against the socket contacts. This allows the flat cable to be easily accommodated and a reliable connection to be provided. The snap connection may comprise at least one snap hook and the cable receiving area may comprise at least one snap projection, wherein the at least one snap hook and the at least one snap projection interlock in the closed state to form the snap connection. Preferably, the snap connection has a plurality of snap hooks and a plurality of snap projections which, in the closed state, are formed in a mirror-symmetrical manner with respect to a plane of symmetry that includes the longitudinal axis of the cable receiving area. The symmetrical arrangement can provide a uniform force distribution across the cable contacts and the socket contacts.

The lid may have a locking clip which extends transversely over the lid and has at least two of the snap hooks, the locking clip being designed to deform elastically in order to exert a force on the contact section in the closed state, by means of which force the cable contacts are pressed against the socket contacts. The locking clip can bulge outward in the closed state, thereby providing reliable pretensioning between the lid and the cable receiving area. This allows the cable contacts and the socket contacts themselves to be reliably contacted over a long period of time, in particular compensating for material fatigue. The lid can have in particular bevels. The bevels can promote elastic deformation of the locking clip. Alternatively, the lid and the at least one snap hook can be formed as a single piece. This allows for easy assembly.

The lid may comprise at least one locking projection and the cable receiving area may comprise at least one locking recess. The at least one locking projection and the at least one locking recess can interlock in the closed state to form the snap connection. This allows the flat cable to be easily accommodated and a reliable connection to be provided. Preferably, the snap connection has a plurality of locking projections and a plurality of locking recesses which, in the closed state, are formed in a mirror-symmetrical manner with respect to a plane of symmetry that includes the longitudinal axis of the cable receiving area. The symmetrical arrangement can provide a uniform force distribution across the cable contacts and the socket contacts. The lid can have at least one slot along the at least one locking projection. The slot can increase elastic deformation of the locking projection. This allows the assembly to be simplified. Preferably, the lid has a plurality of slots along the multiple locking projections. The multiple slots can be formed in the closed state in a mirror-symmetrical manner with respect to a plane of symmetry that includes the longitudinal axis of the cable receiving area.

The endoscope device may further comprise a further flat cable extending through the cable feedthrough from the proximal side to the distal side, wherein the further flat cable on the proximal side comprises a further contact section with at least three further cable contacts arranged one behind the other with respect to a longitudinal axis of the further flat cable, and wherein the socket is further designed to receive the further contact section of the further flat cable. The cable receiving area can also have three further socket contacts, which are arranged one behind the other with respect to the longitudinal axis of the cable receiving area and so as to correspond to the further cable contacts. The lid can also be designed to exert a force on the further contact section in the closed state, by means of which force the further cable contacts are pressed against the further socket contacts. Using a single socket for two or more flat cables allows for efficient use of installation space and simplifies assembly.

The endoscope device may further comprise at least one functional unit located on the distal side of the shaft. The endoscope device may comprise at least one circuit board located on the proximal side of the shaft. The socket can be attached to the circuit board. The flat cable can connect the functional unit to the circuit board. The functional unit can include at least one light source. The light source can be an LED. The functional unit can include at least one camera, in particular a stereo camera. The camera may, for example, have a digital image sensor that has a MIPI interface of version CSI-2 or CSI-3.

The further flat cable can connect the functional unit and/or a further functional unit to the circuit board. The lid and the circuit board may have a screw connection designed to exert a force on the contact section in the closed state, thereby pressing the cable contacts against the socket contacts. The lid and the circuit board may have a screw connection designed to exert a force on the contact section and the further contact section in the closed state, by means of which force the cable contacts and the further cable contacts are pressed against the socket contacts and the further socket contacts. The screw connection between the lid and the circuit board can provide reliable contact. The lid can have at least one lid opening and at least one fastening screw that extends through the at least one lid opening. The cable receiving area can have at least one through-hole for passing through the at least one fastening screw. The circuit board can have at least one threaded hole for forming the screw connection with at least one fastening screw. The threaded hole can be formed by a solderable threaded insert.

The further socket contacts can be arranged laterally offset from the socket contacts, and the screw connection can be arranged between the socket contacts and the further socket contacts. This allows a pretensioning to be applied evenly between the socket contacts and the cable contacts, and between the further socket contacts and the further cable contacts. The lid can be designed to deform elastically through the screw connection in order to exert a force on the contact section and the further contact section in the closed state, by means of which force the cable contacts and the further cable contacts are pressed against the socket contacts and the further socket contacts. The socket may have a gap in the region of the screw connection between the lid and the cable receiving area in the closed state. The lid can be designed in particular to bend through the screw connection in the closed state, in particular to bend into the gap. The lid can be elastically deformed by the screw connection, thereby providing a reliable pretensioning between the lid and the cable receiving area. This allows the cable contacts and the socket contacts themselves to be reliably contacted over a long period of time, in particular compensating for material fatigue.

The electrical connector can further comprise a further flat cable that comprises a further contact section with at least three further cable contacts, wherein the further cable contacts are arranged one behind the other with respect to a longitudinal axis of the further flat cable, and wherein the socket has a lid which is designed to close off the cable receiving area, wherein the lid is further designed to act upon the contact section in a closed state with a force, by means of which the cable contacts are pressed against the socket contacts, and wherein the socket is further designed to receive the further contact section of the further flat cable. The cable receiving area can have at least three further socket contacts, which are arranged one behind the other with respect to the longitudinal axis of the cable receiving area and so as to correspond to the further cable contacts. The lid can also be designed to exert a force on the further contact section in the closed state, by means of which force the further cable contacts are pressed against the further socket contacts. Using a single socket for two or more flat cables allows for efficient use of installation space and simplifies assembly.

The socket can further comprise a lid for a further flat cable, in particular for the electrical connector, which is designed to close off the cable receiving area, wherein the lid is further designed to act upon the contact section in a closed state with a force, by means of which the cable contacts are pressed against the socket contacts, and wherein the socket is further designed to receive a further contact section of the further flat cable. The cable receiving area can have at least three further socket contacts arranged one behind the other with respect to the longitudinal axis of the cable receiving area. The lid can also be designed to exert a force on the further contact section in the closed state, by means of which force further cable contacts of the flat cable are pressed against the further socket contacts. Using a single socket for two or more flat cables allows for efficient use of installation space and simplifies assembly.

The endoscope can be designed as a rigid endoscope. The endoscope may include the endoscope device, a handle, a computing unit, and a connecting cable that connects the computing unit to the endoscope device.

The method for assembling an endoscope device may, following the insertion of the contact section of the flat cable into the cable receiving area of the socket, comprise engaging the first positioning element and the second positioning element. The interlocking action causes the positioning elements to mesh together. The method may subsequently comprise closing the lid of the socket.

The method can be carried out partially or completely manually. In other embodiments, the method can be carried out partially or fully automatically.

The devices according to the invention and the methods according to the invention should not be limited to the application and embodiment described above. In particular, they can have a number of individual elements, components and units as well as method steps, which differ from a number mentioned herein, in order to fulfill a function described herein. In addition, for the ranges of values specified in this disclosure, values within the stated limits shall also be deemed to be disclosed and to be usable in any manner.

It is in particular pointed out that all features and properties described with regard to a device, but also procedures, can be analogously transferred to methods and can be used within the meaning of the invention and are considered to be co-disclosed. The same applies in the opposite direction. This means that structural features mentioned in relation to methods, i.e., features relating to the device, can also be taken into account, claimed and also counted as part of the disclosure within the scope of the device claims.

The present invention is described below by way of example with reference to the accompanying figures. The drawings, the description, and the claims contain numerous features in combination. A person skilled in the art will also, expediently, consider the features individually and use them in combination as appropriate in the context of the claims.

If there is more than one example of a particular object, only one of them may be provided with a reference sign in the figures and in the description. The description of this example can be transferred accordingly to the other examples of the object. If objects are named using number words, such as first, second, third object, etc., these are used to name and/or assign objects.

Accordingly, for example, a first object and a third object may be included but not a second object. However, a number and/or sequence of objects could also be derived using numerical words.

### IN THE DRAWINGS

FIG. 1 is a schematic perspective view of an endoscope;

FIG. 2 is a schematic perspective view of a shaft in a sectional view;

FIG. 3 is a schematic view of a flat cable in a top view;

FIG. 4 is a schematic perspective view of a socket;

FIG. 5 is a schematic view of a two-row flat cable in top view;

FIG. 6 is a schematic perspective view of a two-row socket; and

FIG. 7 is a schematic perspective view of a socket with a pivot joint and snap connection in an open state;

FIG. 8 is a schematic perspective view of the socket with a pivot joint and snap connection in a closed state;

FIG. 9 is a schematic enlarged partial sectional view of the socket with pivot joint and snap connection in an open state;

FIG. 10 is a schematic exploded view of a socket with two locking clips;

FIG. 11 is a schematic perspective view of a socket of an endoscope device with two flat cables in an open state and equipped with a pivot joint;

FIG. 12 is a schematic perspective sectional view of the socket of the endoscope device with two flat cables in a closed state, said being provided with the pivot joint;

FIG. 13 is a schematic perspective view of a socket of an endoscope device with two flat cables in an open state and equipped with a slide-in lid;

FIG. 14 is a schematic perspective sectional view of the socket of the endoscope device with two flat cables in a closed state, said socket being provided with the slide-in lid;

FIG. 15 is a schematic perspective view of a socket of an endoscope device with two flat cables in an open state, said socket being provided with a screw connection;

FIG. 16 is a schematic perspective sectional view of the socket of the endoscope device with two flat cables and provided with a screw connection in a closed state; and

FIG. 17 is a schematic flow chart of a method for assembling an endoscope device.

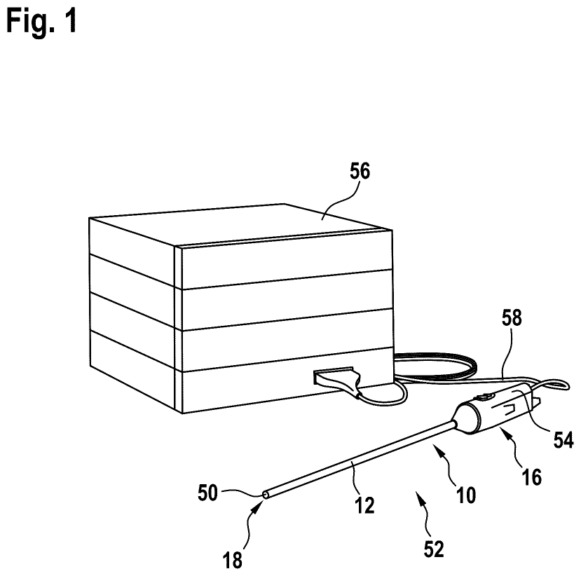

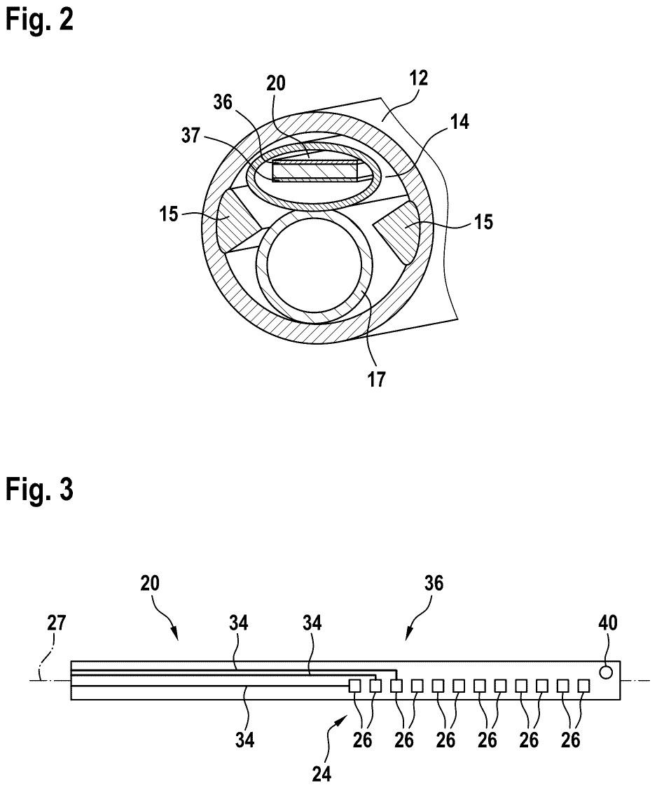

FIG. 1 shows a schematic view of an endoscope 52 with an endoscope device 10. FIG. 2 shows a schematic perspective view of a shaft 12 of the endoscope device 10 in a sectional view.

The endoscope 52 shown by way of example is designed as a rigid endoscope and comprises the endoscope device 10 and a grab handle 54. The endoscope 52 is connected to a supply unit 56 by means of a connecting cable 58. The connecting cable 58 may be part of the endoscope 52. Alternatively, the connecting cable 58 can be part of the supply unit 56. The connecting cable 58 can be detachably connected to the endoscope 52 and/or to the endoscope device 10 and/or to the supply unit 56. The endoscope 52 and/or the endoscope device and the supply unit 56 can be part of a medical system. The supply unit 56 can be connected to a display that is not shown. In addition, the supply unit 56 can be designed to forward and/or process image data received from the endoscope 52.

The shaft 12, shown as rigid by way of example, can be inserted into the interior of organisms or technical cavities with its distal side 18. The endoscope 52 can also be designed as a flexible or partially flexible endoscope with a flexible or partially flexible shaft.

The shaft 12 of the endoscope device 10 comprises a cable feedthrough 14 which extends from a proximal side 16 of the shaft 12 to a distal side 18 of the shaft 12. The cable feedthrough 14 runs inside the shaft 12. The inner width of the cable feedthrough 14 is greater than the inner height of the cable gland 14. The shaft 12 has two stiffening elements 15 extending in the longitudinal direction of the shaft 12, and a structural element 17 extending in the longitudinal direction of the shaft 12. However, this design is to be understood purely as an example. The two stiffening elements 15 are designed to stiffen the shaft 12. In this case, the structural element 17 is designed as a working channel through which, for example, a medical instrument can be guided. The structural element 17 can alternatively also be designed as a further stiffening element, light guide or lens system.

FIG. 3 shows a schematic view of a flat cable 20 of the endoscope device in top view. The flat cable 20 extends through the cable feedthrough 14 from the proximal side 16 to the distal side 18. The flat cable 20 has a contact section 24 with twelve cable contacts 26 on the proximal side 16, this number being purely exemplary.

The flat cable 20 is designed as an FPC cable (flexible printed circuit cable). The flat cable 20 can transmit data from a digital image sensor, for example via a MIPI interface of version CSI-3. The flat cable 20 has twelve conductor tracks 34, which extend from each of the cable contacts 26 away from the contact section 24 along a longitudinal axis 27 of the flat cable 20.

The flat cable 20 is designed as a two-layer flat cable. However, depending on the width of the conductor tracks and the number of signals, it can also be implemented on 3 or 4 layers. The conductor tracks 34 run in two, three or four different cable layers, a first cable layer 36 and a second cable layer 37, of which only the first cable layer 36 is shown in FIG. 3, which is an upper cable layer. Cable layers 36, 37 are shown schematically in FIG. 2. In this case, conductor tracks for power supply run in the first cable layer 36 and conductor tracks for signal transmission run in the second cable layer 37. The flat cable 20 is not limited to two cable layers, but can, for example, also have three or four cable layers, each of which can be designed for data and/or power transmission.

The contact section 24 of the flat cable 20 is designed to electrically connect the flat cable 20 to a socket 28. The contact section 24 has twelve cable contacts 26. The contact section 24 has a width that is smaller than the inner width of the cable feedthrough 12 and a height that is smaller than the inner height of the cable feedthrough 12. In this case, the contact section 24 changes the course of the conductor tracks 34 from a direction along the longitudinal axis of the flat cable 20 to a direction transverse to the longitudinal axis 27 of the flat cable 20. The cable contacts 26 are electrically connected to the conductor tracks 34. The cable contacts 26 are arranged offset from and spaced apart with respect to the longitudinal axis 27 of the flat cable 20, in other words one behind the other.

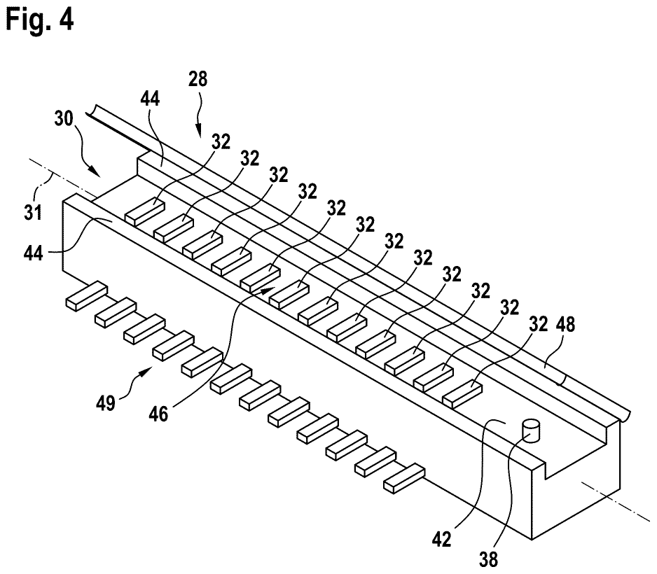

The socket 28 provides, as shown in FIG. 4, an electrical connection to the cable contacts 26, and makes this available to a circuit board (not shown) of the endoscope device 10, on which circuit board the socket 28 may be attached. The socket 28 has a cable receiving area 30 with twelve socket contacts 32, where the number of socket contacts 32 corresponds to the number of cable contacts 26. The socket contacts 32 are offset from and spaced apart with respect to a longitudinal direction 31 of the cable receiving area 30, in other words one behind the other. The socket contacts 32 are arranged in the same pattern as the cable contacts 26. The socket contacts 32 are designed and/or arranged to correspond to the cable contacts 26. The socket 28 also has twelve protruding contacts 49 which provide an electrical connection with the circuit board.

The cable receiving area 30 of the socket has a first positioning element 38. The contact section 24 of the flat cable 20 has a second positioning element 40. The first positioning element 38 and the second positioning element 40, working together, cause the contact section 24 to be placed in the cable receiving area 30. The first positioning element 38 is designed as a positioning pin. The second positioning element 40 is designed as a receptacle as a feedthrough that can accommodate the positioning pin. The first positioning element 38 can be inserted into the second positioning element 40.

The cable receiving area 30 has a base 42 and two side walls 44. The two side walls 44 together define a receiving space 46 for the contact section 24 of the flat cable 20. The side walls 44 each extend parallel to a longitudinal axis of the cable receiving area 30. The socket 28 has a lid 48 which can close off the cable receiving area 30. The lid 48 exerts a force on the contact section 24 in a closed state, by means of which force the cable contacts 26 are pressed against the socket contacts 32. The cable contacts 26 of the cable 20 can be inserted from a transverse direction of the socket 28.

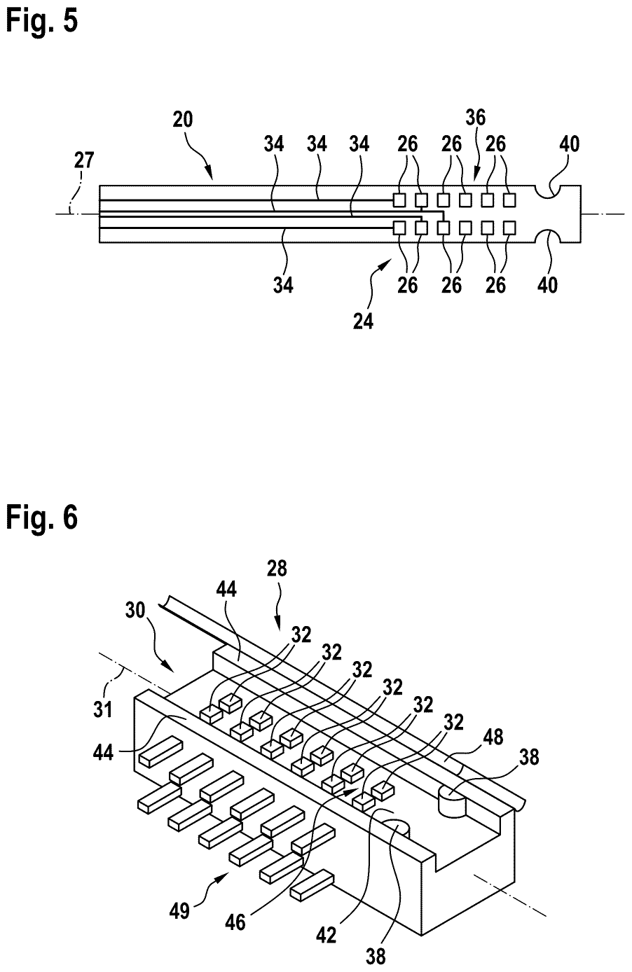

FIG. 5 shows a schematic view of a two-row flat cable 20 as an alternative embodiment of the flat cable 28 described above in top view. FIG. 6 shows a schematic perspective view of a two-row socket 28 as an alternative embodiment of the socket 28 described above. The two-row flat cable 20 largely corresponds to the previously described single-row flat cable 20 shown in FIG. 3, wherein a contact section 24 is designed as a double row. The two-row socket 28 largely corresponds to the single-row socket 28 described above and shown in FIG. 4, wherein a cable receiving area 30 is designed in two rows. Regarding the description, reference can also be made to the above statements.

The two-row contact section 24 of the two-row flat cable 20 is designed to electrically connect the two-row flat cable 20 to the two-row socket 28. In addition, conductor tracks 34 run in three different cable layers, of which only a first cable layer 36 is shown in FIG. 5, which is an upper cable layer. The contact section 24 has twelve cable contacts 26 arranged in two rows, where six cable contacts 26 are arranged one behind the other with respect to a longitudinal axis 27 of the two-row flat cable 20. The cable contacts 26 are therefore arranged in two rows offset from and spaced apart with respect to the longitudinal axis 27 of the two-row flat cable 20, in other words arranged in two rows one behind the other.

The two-row cable receiving area 30 of the two-row socket 28 has twelve socket contacts 32, which are arranged in two rows, wherein six socket contacts 32 are arranged one behind the other with respect to a longitudinal axis 31 of the cable receiving area 30 of the two-row socket 28 and so as to correspond to the cable contacts 26. The socket contacts 32 are therefore arranged in two rows offset from and spaced apart with respect to a longitudinal direction 31 of the cable receiving area 30, in other words arranged in two rows one behind the other. The socket contacts 32 are arranged in the same pattern as the cable contacts 26. The number of socket contacts 32 corresponds to the number of cable contacts 26. The two-row socket 28 also has twelve protruding contacts 49 which provide an electrical connection with the circuit board. The twelve protruding contacts 49 of the two-row socket 28 are arranged on two different sides of the two-row socket 28, with only six of the twelve protruding contacts 49 on one side being shown in FIG. 6.

The cable receiving area 30 of the two-row socket 28 has, as shown in FIG. 5, a first positioning element 38. The contact section 26 of the two-row flat cable 20 has, as shown in FIG. 6, a second positioning element 40. The first positioning element 38 and the second positioning element 40 together cause the contact section 24 of the two-row flat cable 20 to be placed in the cable receiving area 30 of the two-row socket 28. The first positioning element 38 comprises two positioning ribs and the second positioning element 40 comprises two notches designed to accommodate the two positioning ribs.

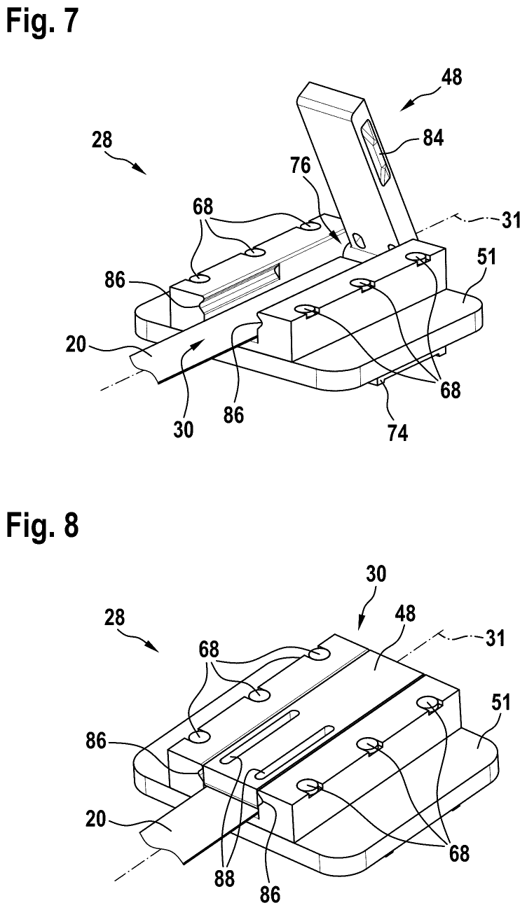

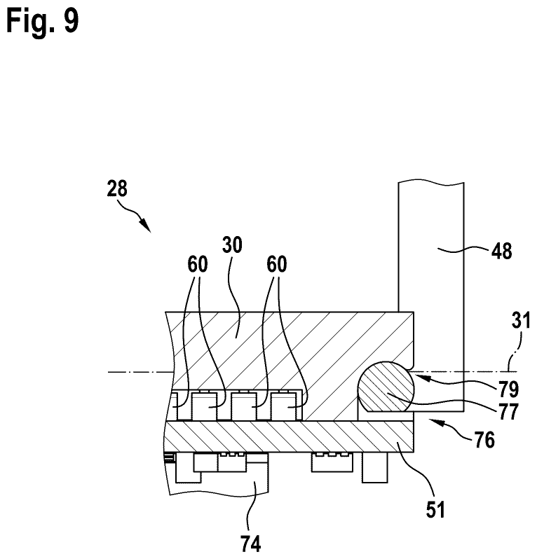

FIG. 7, FIG. 8 and FIG. 9 show a socket 28 with a pivot joint and snap connection as a further embodiment of the socket 28 described above. FIG. 7 shows a schematic perspective view of the socket 28 with pivot joint and snap connection in an open state and FIG. 8 shows a schematic perspective view of the socket 28 with pivot joint and snap connection in a closed state. FIG. 9 shows a schematic enlarged sectional view of the socket 28 with pivot joint and snap connection in an open state. FIG. 7 and FIG. 8 also show a flat cable 20 which largely corresponds to the previously described single-row flat cable 20 shown in FIG. 3. The socket 28 is shown in a state assembled on a circuit board 51. The socket 28 is assembled on one side of the circuit board 51 by means of six fastening bolts 68. The circuit board 51 has two socket strips 74 on the opposite side for a plug connection to the power supply unit 56 via the connecting cable 58.

The socket 28 with pivot joint and snap connection corresponds functionally to the previously described, single-row socket 28 shown in FIG. 4, wherein a closing process with the lid 48 is designed differently. The cable receiving area 30 and the lid 48 are connected via a pivot joint 76. One axis of rotation of the pivot joint 76 runs transversely to the longitudinal axis 31 of the cable receiving area 30. Regarding the description, reference can also be made to the above statements.

The lid 48 and the cable receiving area 30 have a snap connection which, in the closed state, is designed to exert a force on the contact section 24, by means of which force the cable contacts 26 (not shown) are pressed against the socket contacts 32. The lid 48 has two locking projections 84 and the cable receiving area 30 has two locking recesses 86, which interlock in the closed state to form the snap connection. However, the flat cable can be easily accommodated and a reliable connection can be provided. The lid 48 has two slots 86, each running along a locking projection 86 to increase elastic deformation of the locking projection for easy assembly. The two locking projections 84, the two locking recesses 86 and the two slots 86 are formed in the closed state in a mirror-symmetrical manner with respect to a plane of symmetry that includes the longitudinal axis 31 of the cable receiving area 30 in order to provide a uniform force distribution over the cable contacts 26 (not shown) and the socket contacts 32

The pivot joint 76 shown in FIG. 9 is designed as a detachable hinge. The pivot joint 76 has two flattened pins 77 arranged on opposite sides of the lid 48 and two openings 79 arranged on opposite sides of the cable receiving area 30 in which the flattened pins 77 are rotatably received. The flattened pins 77 allow removal from the two openings 79 to provide a detachable connection between the lid 48 and the cable receiving area 30. For example, the lid 48 can be replaced if it malfunctions.

The socket contacts 32 of the socket 28 shown in FIG. 9 are designed as spring contacts, in particular as contact springs, in order to exert a force on the contact section 24 in the closed state. This allows for reliable contact between cable contacts 28 (not shown) and socket contacts 32.

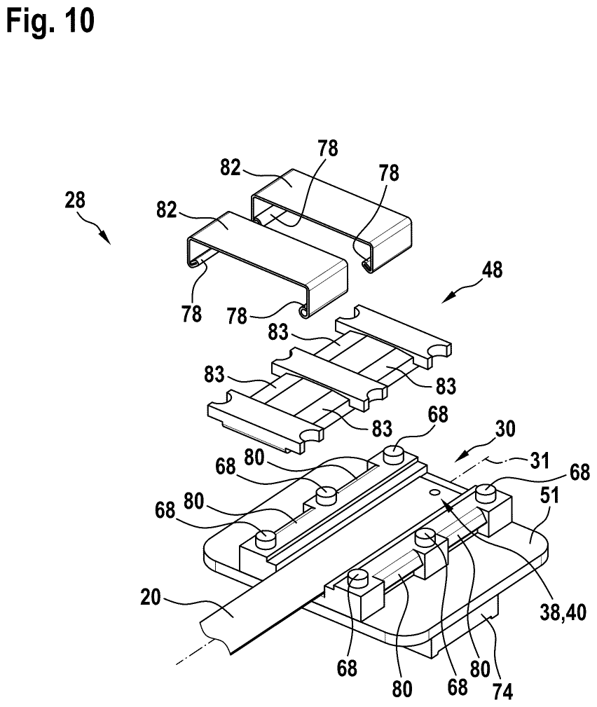

FIG. 10 shows a schematic exploded view of a socket 28 with two locking clips as a further embodiment of the socket 28 described above. The socket 28 with two locking clips largely corresponds to the socket 28 with pivot joint and snap connection described above and shown in FIGS. 7, 8 and 9, wherein no pivot joint is provided and the snap connection is designed differently. Regarding the description, reference can also be made to the above statements.

The socket 28 shown in FIG. 10 has a snap connection with four snap hooks 78, which are arranged in pairs on two locking clips 82 of the lid 48, and four snap projections 80, which are arranged in pairs on the cable receiving area 30. The snap hooks 78 and the snap projection 80 interlock in the closed state to form the snap connection. The two locking clips 82 each extend transversely over the lid 48 and are designed to deform elastically in order to exert a force on the contact section 24 in the closed state, by means of which force the cable contacts 28 are pressed against the socket contacts 32. The locking clip 82 bulges outward during elastic deformation in the closed state. The lid 48 has four bevels 83 which facilitate the bulging of the locking clips 82. This allows the lid 48, in the closed state, to exert a force on the contact section 24, by means of which force the cable contacts 28 of the flat cable 20 (not shown) are pressed against the socket contacts 32 (not shown). Due to the elastic deformation of the locking clip 82, force can be reliably applied even over a long period of time, thereby compensating for material fatigue in particular. The positioning of the flat cable 20 in the cable receiving area 30 is carried out by the first and second positioning elements 38, 40 described above. The lid 48 with the two locking clips 82 is, in the closed state, mirror-symmetrical to a plane of symmetry that includes the longitudinal axis 31 of the cable receiving area 30.

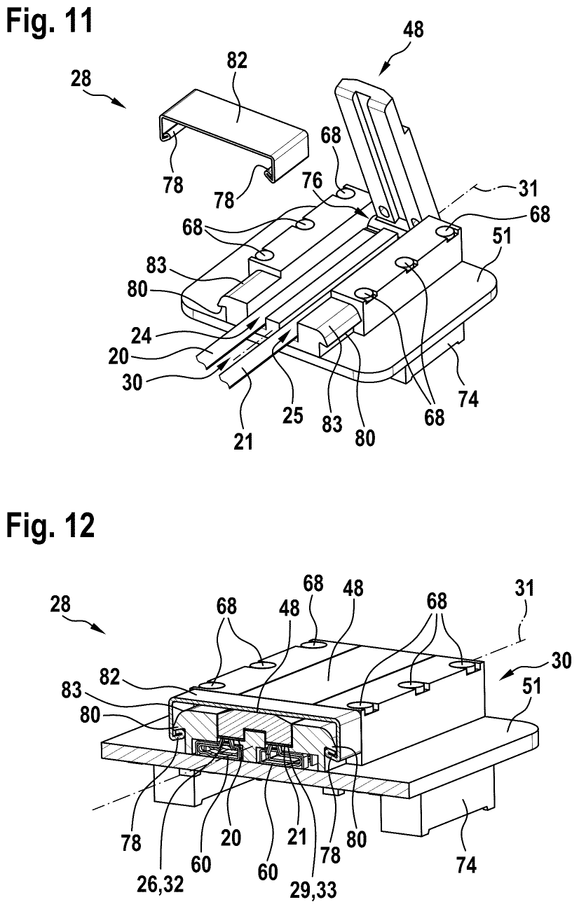

FIG. 11 and FIG. 12 show a socket 28 of an endoscope device 10 with a pivot joint and two flat cables 20, 21 as a further embodiment of the endoscope device 10 described above. The endoscope device 10 with two flat cables 20, 21 largely corresponds to the endoscope device 10 described above and shown in FIG. 1, wherein a further flat cable 21 extends through the cable feedthrough 14 from the proximal side 16 to the distal side 18. The cable feedthrough 14 largely corresponds to the cable feedthrough 14 described above and shown in FIG. 2, wherein the further flat cable 21 together with the flat cable 20 extends through the cable feedthrough 14 from the proximal side 16 to the distal side 18. The flat cable 20 and the further flat cable 21 each correspond largely to the previously described, single-row flat cable 20 shown in FIG. 3, wherein the arrangement of the second positioning element 40 is different. The socket 28 for two flat cables 20, 21 corresponds functionally to the socket 28 with two locking clips described above and shown in FIG. 10, wherein it is designed to accommodate a further flat cable 21 and a closing process of the cable receiving area 30 with the lid 48 is designed differently.

The further flat cable 21 has on the proximal side 16 a further contact section 25 with twelve further cable contacts 29, which are arranged one inside the other with respect to a longitudinal axis of the further flat cable 21. The socket 28 is further equipped to receive the further contact section 25 of the further flat cable 21, wherein the cable receiving area 30 has twelve further socket contacts 33 which are arranged one behind the other with respect to the longitudinal axis 31 of the cable receiving area 30 and so as to correspond to the further cable contacts 29. The lid 48 is further designed to exert a force on the further contact section 25 in the closed state, by means of which force the further cable contacts 29 are pressed against the further socket contacts 33. The socket 28 has the lid 48, which is connected to the cable receiving area 30 via a pivot joint 76. The pivot joint 76 corresponds to the pivot joint described above and shown in FIG. 9.

FIG. 11 shows a schematic perspective view of the socket 28 of the endoscope device 10 with the pivot joint 76 and two flat cables 20, 21 in an open state. FIG. 12 shows a schematic perspective sectional view of the socket 28 of the endoscope device 10 provided with the pivot joint 76 with two flat cables 20, 21 in a closed state. Regarding the description, reference can also be made to the above statements.

The lid 48 and the cable receiving area 30 have a snap connection which, in the closed state, is designed to exert a force on the contact section 24 and the further contact section 25, by means of which force the cable contacts 26 and the further cable contacts 29 are pressed against the socket contacts 32 and the further socket contacts 33. The snap connection comprises two snap hooks 78, which are arranged on the locking clip 82 of the lid 48, and two snap projections 80, which are arranged on the cable receiving area 30 and interlock with the two snap hooks 78 in the closed state. Regarding the description of the snap connection with the locking clip 82, reference can also be made to the above explanations. The socket contacts 32 and the further socket contacts 33 are formed as spring contacts 60, in particular as contact springs. In an alternative design, for example, the locking clip 82 can be replaced by snap hooks that are formed as a single piece on a lid.

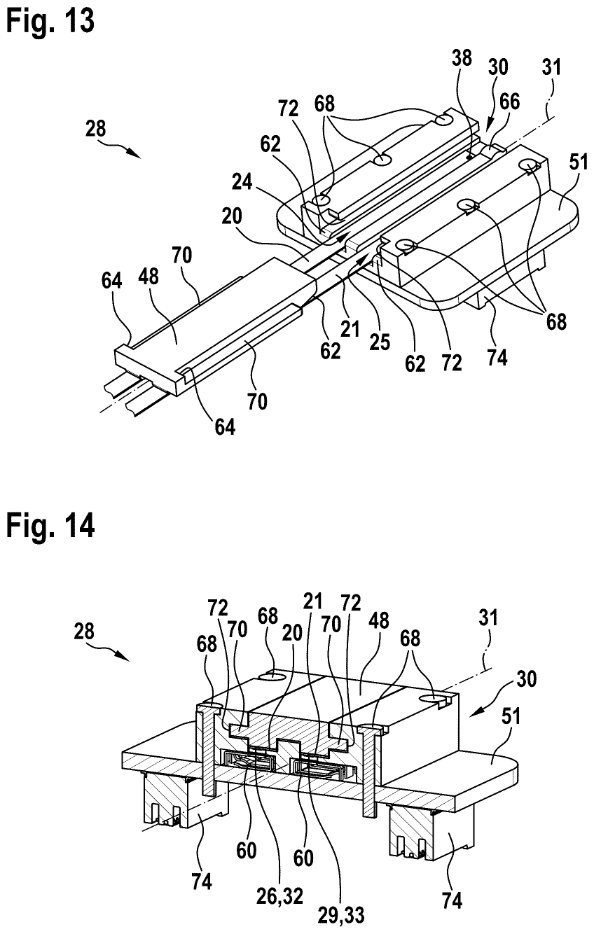

FIG. 13 and FIG. 14 show a socket 28 of the endoscope device 10 with a slide-in lid 48 and two flat cables 20, 21 as a further embodiment. The socket 28 of the endoscope device 10 with two flat cables 20, 21, which is provided with the slide-in lid 48, largely corresponds to the socket 28 of the endoscope device 10 with two flat cables 20, 21 described above and shown in FIG. 11 and FIG. 12, which socket is provided with the pivot joint 76, wherein a closing process of the cable receiving area 30 with the lid 48 is designed differently. FIG. 13 shows a schematic perspective view of the socket 28 of the endoscope device 10 with the slide-in lid 48 and two flat cables 20, 21 in an open state. FIG. 14 shows a schematic perspective sectional view of the socket 28 of the endoscope device 10 with the slide-in lid 48 and two flat cables 20, 21 in a closed state.

The lid 48 and the cable receiving area 30 can be slid into each other to close off the cable receiving area 30. The lid 48 has two lid projections 70 and the cable receiving area 30 has two receptacle recesses 72, wherein the lid projections 70 and the receptacle recesses 72 can be slid into each other to close the cable receiving area 30. The lid 48 can be pushed into the cable receiving area 30 by inserting it along the longitudinal direction 31 to achieve the closed state shown in FIG. 14. The lid 48 and the cable receiving area 30 have insertion bevels 62 to make inserting the lid 48 easy and smooth during assembly. The lid 48 also has an end stop 64 to reliably achieve the closed state. Additionally, the socket 32 has a locking mechanism in which a locking projection 66 and a locking recess (not shown) interlock in the closed state. The locking mechanism prevents movement from the closed state and thus allows secure fixing. The socket contacts 32 and the further socket contacts 33 are each formed as spring contacts 60, in particular as contact springs. In the closed state, the lid 48 exerts a force on the contact section 24 and the further contact section 25, by means of which force the cable contacts 26, 29 are pressed against the socket contacts 32, 33 that are designed as spring contacts 60.

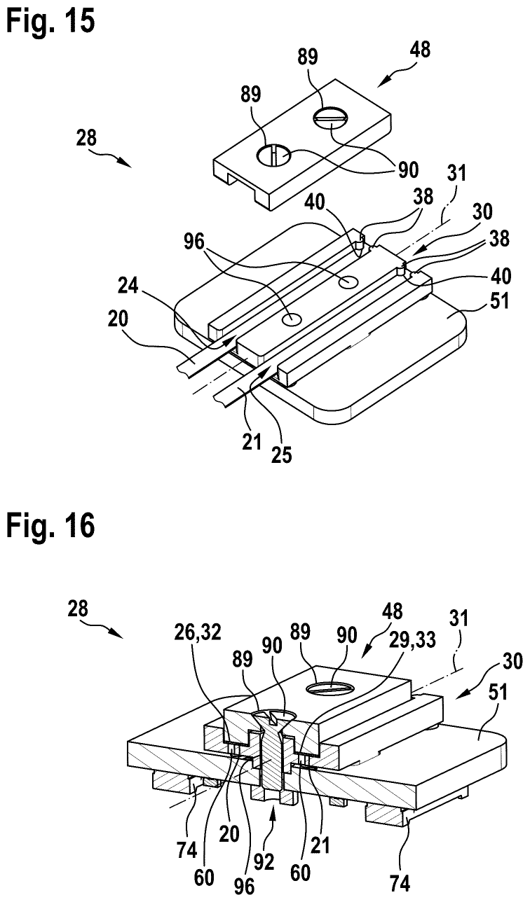

FIG. 15 and FIG. 16 show a socket 28 of an endoscope device 10 with a screw connection and two flat cables 20, 21 as a further embodiment. The socket 28 of the endoscope device 10 with two flat cables 20, 21 which is provided with a screw connection largely corresponds to the socket 28 of the endoscope device 10 with two flat cables 20, 21 described above and shown in FIG. 13 and FIG. 14, which socket is provided with the slide-in lid 48, wherein a closing process of the cable receiving area 30 with the lid 48 is designed differently.

The lid 48 and the circuit board 51 have a screw connection which, in the closed state, is designed to exert a force on the contact section 24 and the further contact section 25, by means of which force the cable contacts 26 and the further cable contacts 29 are pressed against the socket contacts 32 and the further socket contacts 33. The screw connection between the lid 48 and the circuit board 51 provides reliable contact between the flat cables, 20, 21 and the socket 28. The socket contacts 32 and the further socket contacts 33 are each formed as spring contacts 60, in particular as spring pins.

The lid 48 has two lid openings 89 and two fastening screws 90, each of the screws extending through one of the two lid openings 89. The cable receiving area 30 has two through holes 96 for passing through the two fastening screws 90. The circuit board 51 has two threaded holes 92 for forming the screw connection with the two fastening screws 90. The two threaded holes 92 are formed by solderable threaded inserts. The flat cables 20, 21 are positioned with respect to the cable receiving area 30 by means of a first positioning element 38, which is formed as two positioning ribs on the cable receiving area 30, and a second positioning element 40, which is formed as two notches on the flat cables 20, 21.

The further socket contacts 33 are arranged laterally offset from the socket contacts 32, and the screw connection is arranged therebetween. The lid 48 is further designed to deform elastically through the screw connection, in the closed state, in order to exert a force on the contact section 24 and the further contact section 25, by means of which force the cable contacts 26 and the further cable contacts 29 are pressed against the socket contacts 32 and the further socket contacts 33. The socket 28 has a gap 94 in the region of the screw connection between the lid 48 and the cable receiving area 30 in the closed state, and the lid 48 is further arranged to deflect through the screw connection in the closed state, in particular to deflect into the gap 94. The lid 48 can be elastically deformed by the screw connection, thereby providing a reliable pretensioning between the lid 48 and the cable receiving area 30. This allows the cable contacts 26, 29 and the socket contacts 32, 33 to be reliably contacted even over a long period of time, thereby compensating in particular for material fatigue.

The flat cable 20 and the socket 28 together form an electrical connector for an endoscope device 10.

The endoscope device 10 has a functional unit 50 which is located on the distal side of the shaft 12. The present functional unit 50 comprises a camera with a digital image sensor that has a MIPI interface of version CSI-3. In some embodiments, the functional unit 50 alternatively or additionally comprises at least one light element such as an LED, for example one or more white light LEDs and/or one or more colored LEDs. The circuit board of the endoscope device 10, which is not shown, is located on the proximal side of the shaft 12. The socket 12 is attached to the circuit board with its protruding contacts 49 to provide an electrical connection with the circuit board. The flat cable 20 connects the functional unit 50 to the circuit board. The circuit board can in turn be connected to the power supply unit 56. The further flat cable 21 can connect the functional unit 50 and/or a further functional unit to the circuit board.

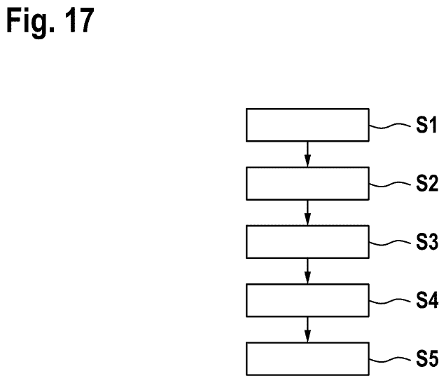

The foregoing description shows how the endoscope device 10 is assembled in a method for assembling an endoscope device. FIG. 17 shows a schematic flow chart of a method for assembling an endoscope device 10. The method comprises step S1 of providing a shaft 12 with a cable feedthrough 14 extending from a proximal side 16 of the shaft 12 to a distal side 18 of the shaft 12. Furthermore, the method comprises a step S2 of providing a flat cable 20, wherein the flat cable 20 comprises a contact section 24 with at least three cable contacts 26 on the proximal side 16, the cable contacts 26 being arranged one behind the other with respect to a longitudinal axis 27 of the flat cable 20. Furthermore, the method comprises a step S3 of providing a socket 28 on the proximal side 16 of the shaft 12, wherein the socket 28 comprises a cable receiving area 30 designed to receive the contact section 24 of the flat cable 20, wherein the cable receiving area 30 has at least three socket contacts 32 arranged one behind the other with respect to a longitudinal axis 31 of the cable receiving area 30 and so as to correspond to the cable contacts 26. Furthermore, the method comprises a step S4 of passing the flat cable 20 through the cable feedthrough 14. Furthermore, the method comprises step S5 of inserting the contact section 24 of the flat cable 20 into the cable receiving area 30 of the socket 28.

### LIST OF REFERENCE SIGNS

10 endoscope device
12 shaft
14 cable feedthrough
15 retaining element
16 proximal side
17 structural element
18 distal side
20 flat cable
21 flat cable
24 contact section
25 contact section
26 cable contacts
27 longitudinal axis
28 socket
29 cable contacts
30 cable receiving area
31 longitudinal axis
32 socket contact
33 socket contact
34 conductor track
36 first cable layer
37 second cable layer
38 first positioning element
40 second positioning element
42 base
44 side wall
46 receiving space
48 lid
49 protruding contacts
50 functional unit
51 circuit board
52 endoscope
54 grab handle
56 supply unit
58 connecting cable
60 spring contact
62 insertion bevel
64 end stop
66 locking projection
68 fastening bolt
70 lid projection
72 receptacle recess
74 socket strip
76 pivot joint
77 pin
78 snap hook
79 opening
80 snap projection
82 locking clip
83 bevel
84 locking projection
86 locking recess
88 slot
89 lid opening
90 fastening screw
92 threaded hole
94 gap
96 through-hole

1-40. (canceled)

41. An endoscope, comprising:
a shaft comprising a cable passage extending from a proximal side of the shaft to a distal side of the shaft;
a flat cable extending through the cable passage from the proximal side to the distal side, wherein the flat cable comprises a contact section with at least three cable contacts its proximal side, wherein the cable contacts are arranged one behind the other with respect to a longitudinal axis of the flat cable; and
a socket arranged on the proximal side of the shaft and comprising a cable receptacle adapted to receive the contact section of the flat cable, wherein the cable receptacle has at least three socket contacts which are arranged one behind the other with respect to a longitudinal axis of the cable receptacle and correspond to the cable contacts.
